(19) United States
(12) Patent Application Publication
(10) Pub. No.: US 2024/0058497 A1
(43) Pub. Date: Feb. 22, 2024

(54) ULTRA VIOLET C SELF-STERLIZING GERMICIAL LIGHT-EMITTING DOOR HANDLE/LEVER ACCESS DEVICE AND ALIKE FOR HAND SANITATION

(71) Applicant: Tom Chi, Wichita, KS (US)

(72) Inventor: Tom Chi, Wichita, KS (US)

(21) Appl. No.: 18/269,534

(22) PCT Filed: Jun. 7, 2022
(86) PCT No.: PCT/US2022/032551

§ 371 Date: Jun. 23, 2023

Related U.S. Application Data
(60) Provisional application No. 63/197,576, filed on Jun. 7, 2021.

(51) Int. Cl. A61L 2/26; E05B 1/00; A61L 2/10; A61L 2/00

(52) U.S. Cl. CPC A61L 2/26; E05B 1/0069; A61L 2/10; A61L 2/0047; A61L 2202/11

(57) ABSTRACT

A rotating successive firing emitting translucent ultraviolet C self-sterilizing germicidal light tube for a door handle or door lever access device for hand sanitation, includes a tube handle attached to a door latch assembly, a rotating UVC LED light source contained within the tube handle; electrical energy for energizing the UVC LED light source, electrical energy for rotating the UVC LED light source contained within the tube handle, and a device for rotating the UVC LED light source contained within the tube handle.

### BACKGROUND OF THE INVENTION

### Technical Field of the Invention

The present invention relates generally to disinfecting and sterilizing door access devices such as door handles and alike that are touched by a user's hand by incorporating into the door access device a source of ultraviolet C self-sterilizing germicidal light.

It is well established that germs from hands are easily communicated to a person's face especially when the hands touch the face several times a minute, as for example the eyes are rubbed, the nose is touched and other times when the hands consciously and unconsciously touch the face.

Since the exposed face is more than any other part of the human body more susceptible to transmitting germs to the body's systems it imperative to maintain as best as possible a germ-free face and hands-free of pathogens which can enter the eyes, ears, mouth, nose, etc.

### Description of the Prior Art

The prior art includes UV sanitizing patents that includes Visco et al., U.S. Pat. No. 4,856,140, issued Aug. 15, 1989; Manson et al., U.S. Pat. No. 6,289,557, issued Sep. 18, 2001; Jones, U.S. Pat. No. 7,175,807, issued Feb. 13, 2007; Sassoon, U.S. Pat. No. 7,360,674, issued Apr. 22, 2008; Stropkay et al., U.S. Pat. No. 7,458,742, issued Dec. 2, 2008; Jones, U.S. Pat. No. 7,598,501, issued Oct. 6, 2009; Zevallos, U.S. Pat. No. 7,716,789, issued May 18, 2010; Balzano, U.S. Pat. No. 8,353,085, issued Jan. 15, 2013; Caron, U.S. Pat. No. 8,375,521, issued Feb. 19, 2013; Bracey, U.S. Pat. No. 9,051,757, issued Jun. 9, 2015; Brennan, U.S. Pat. No. 9,200,771, issued Dec. 1, 2015; Roberts et al., U.S. Pat. No. 9,376,833, issued Jun. 28, 2016; and Strombeck et al., U.S. Pat. No. 10,422,158, issued Sep. 24, 2019.

While these patents may be suitable for the purposes for which they were designed, they would not be as suitable for the purposes of the present invention as heretofore described. It is thus desirable to provide a system to prevent contamination of the face and the implications of transmitting germs to the face.

### SUMMARY OF THE INVENTION

A primary object of the present invention is to provide a system for sanitizing door access devices such as door handles to prevent transmission of pathogens from door handles to a person's hand and subsequently to a person's face.

Another object of the present invention is to provide a means to disinfect the door handles for the prevention of pathogens to contact a person and spread disease and other unsanitary conditions.

Another object of the present invention is to utilize LED as a source of ultraviolet C self-sterilizing germicidal light for hand sanitization.

### Rotating Motor

The instant invention provides a rotating UVC LED motor and power transfer to printed circuit board for ultraviolet C self-sterilizing germicidal light to doorknobs, door levers, door pulls and other devices reducing required LEDs by 75%.

Producing a uniform irradiance map for surface decontamination through quartz from inside a device such as our door lever and other devices means that the projection angles of the LEDs will require for example, 24 or more LEDs for some door levers and 36 or more for a door pull.

By utilizing the rotating printed circuit board with LEDs on it we're able to reduce the number of LEDs by ¾ and still achieve the required dosage.

A rotating motor system places a small motor inside of the door lever handle which connects to a power source and the printed circuit boards which make the LEDs function.

The device may be programmed for LEDs to irradiate at any desired time and as well the speed of the motor and rotations may be varied as desired.

Importantly, small motors to achieve this effect are readily available for purchase and implementation and the invention uses the motor and a unique connection to the circuit board and electronics.

### Successive Firing

If desired, successive firing of the LEDS permits conservative peak amperage demands. For example, a door lever with 6 UVC LEDs requires 4 w/LED or 24 W. By using 2 “strings” of LEDs permits turning-on 3 LEDs at a time then successively the other 3 LEDs. Accordingly, only 12 watts are needed at any time and is particularly useful for limiting volume of power needed at any time when using batteries as the source.

### Frosted Appearance

Invention provides a method for obtaining a ‘frosted’ or “milky” appearance to quartz using a clear factory quartz product and then modifying it with engraving. “Milky” quartz is currently available however sizes are limited and it's expensive and the method allows a logo to be present on our ‘milky’ quartz. The resultant product is both aesthetic in that it appears pleasing. As well, the milky or frosted surface conceals the internal electronics of the device so that it doesn't appear “robotic.” Engraving is inexpensive and allows us to use most any custom size with rapid frosted appearance easily and economically achievable. This method also enables a type of “echoing” wherein even a single LED UVC chip may be enabled to irradiate the entire surface of the door lever by internally ‘bouncing’ from refraction created by the milky or frosted surface. The ‘echoing’ then allows a reduction in the amount of LED UVC chips need to achieve sterilization of the quartz surface and accomplishes a major reduction in amperage needed to power the device and as well results in a significant cost reduction.

### Wall Wort Recharging System

Provided is a wall wort recharging system for batteries for ultraviolet C sterilizing germicidal light door accessories such as door levers, door push/pull devices etc. Our previous patents reference batteries as power source for the devices. Our power-transfer systems allow for power without batteries. Our wall wort 9 v (and other voltages) plug-in jack allows for rapidly recharging batteries without physical removal of batteries. The within described wall wort system can also be used as easier than AC hard-wiring yet a quick and easy install that can be made permanent without our snap-together DC track systems.

The foregoing and other objects and advantages will appear from the description to follow. In the description reference is made to the accompanying drawing, which forms a part hereof, and in which is shown by way of illustration specific embodiments in which the invention may be practiced.

Other objects and features of the present invention will become apparent when considered in combination with the accompanying drawing figures, which illustrate certain preferred embodiment of the present invention. It should, however, be noted that the accompanying drawing figures are intended to illustrate only select preferred embodiments of the claimed invention and are not intended as a means for defining the limits and scope of the invention.

### BRIEF DESCRIPTION OF THE DRAWING FIGURES

In the drawing figures, wherein similar features are denoted with similar reference numerals throughout the several views:

FIG. 1 is a perspective view of the ultraviolet C self-sterilizing germicidal light emitting door handle/lever access device for hand sanitation as it would be installed on both sides of a door with the door omitted;

FIG. 2 is another perspective view of the ultraviolet C self-sterilizing germicidal light emitting door handle/lever access device for hand sanitation shown as it would be conventionally installed on both sides of a door with door omitted;

FIG. 3 is a perspective view of one side the ultraviolet C self-sterilizing germicidal light emitting door handle/lever access device for hand sanitation;

FIG. 4 is a partial perspective view of the ultraviolet C self-sterilizing germicidal light emitting door handle/lever access device for hand sanitation illustrating the spinning UVC LED board containing the UVC LED light source;

FIG. 5 is an exploded view of the ultraviolet C self-sterilizing germicidal light emitting door handle/lever access device for hand sanitation;

FIG. 6 is another exploded view of the ultraviolet C self-sterilizing germicidal light emitting door handle/lever access device for hand sanitation;

FIG. 7 shows the successive firing of the UVC LEDs in the rotating tube handles;

FIG. 8 is a perspective partial view illustrating a frosted quartz translucent tube handle with the entire surface engraved, except for a logo, thereby obscuring the electronics within the quartz tube from viewer which will be a saving over a much more costly “milky” quartz;

FIG. 9 is a perspective partial view illustrating high intensity, narrow LED projections;

FIG. 10 illustrates electrical power being transmitted from a door jamb to the motor that rotates the spinning UVC LEF board;

FIG. 11 illustrates the electrical communication from the door jamb to the motor;

FIG. 12 is partial perspective view of the electrical connections on the door displaced from the door handle;

FIG. 13 illustrates a battery powered door handle/lever with a removable cover that covers the requisite battery which powers the rotating tube handles;

FIG. 14 depicts the battery with its cover removed; and,

FIG. 15 illustrates batteries affixed to the door handle.

### DESCRIPTION OF THE REFERENCE NUMERALS

10 door handle latch assembly of invention in place on door
10a door jamb in wall 10b door
12 first translucent door tube handle with internal rotating UVC LED board
12a second translucent door tube handle with internal rotating UVC LED board
14 end plug with aperture to engage with first end of translucent tube to accept projection from first end of rotating UVC LED circuit board
14a projection 14a at first end of rotating UVC LED circuit board
14b projection 14b at second end of rotating UVC LED circuit board
16 flange at second end of translucent tube
18 rotating UVC LED circuit board with attached sequencing UVC LED light source
18a successive firing UVC LED light source
20 UVC LED board connector to motor
22 slip rings for rotating electrical connections
24 electric motor for rotating UVC LED board
26 coupling/adapter housing containing components
28 control board
30 power contact frame
32 successive firing of numbers 2, 4 and 6 UVC LEDs
34 successive firing of numbers 1, 3 and 5 UVC LEDs
36 frosted quartz tube
38 high intensity narrow LED projections
40 electrical contact(s) on door handle assembly
42 electrical contact(s) in door jamb assembly
44a electrical distribution junction
44 battery cover of door handle assembly
46 battery on door handle assemble with cover removed
48 another preferred embodiment of battery on door handle assembly
50 electrical conduits on door
52 electrical conduits on door jamb
54 electrical source, typically 120-volt AC current

### DETAILED DESCRIPTION OF THE DRAWING FIGURES AND PREFERRED EMBODIMENTS

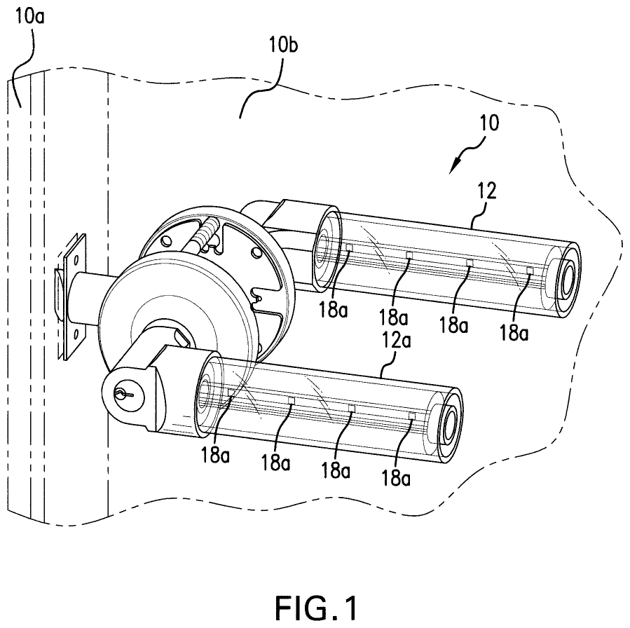

Turning now, in detail, to the accompanying drawing figures, FIG. 1 illustrates the invention 10 assembled to door 10b (shown in phantom) with translucent door tube handle 12 on one side of a door and a second translucent door tube handle 12a on the other side of door 10b. Door jamb 10a is illustrated in phantom.

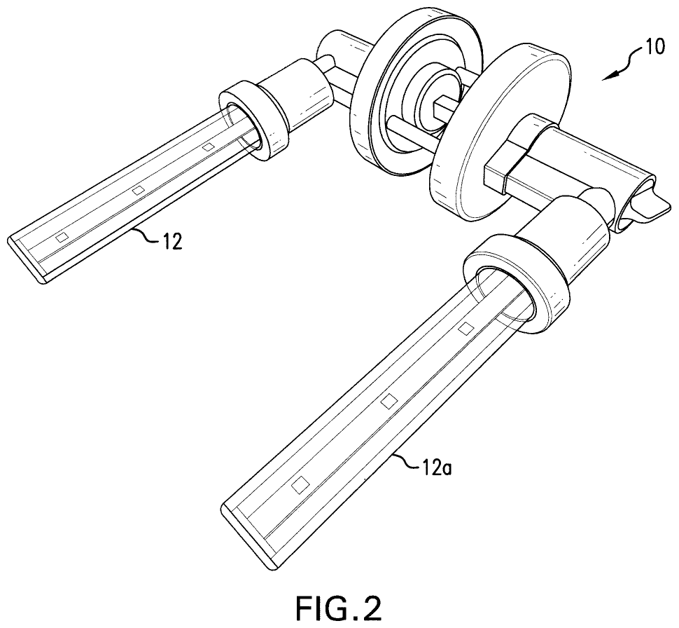

FIG. 2 partially illustrates invention 10 with two translucent door handles, namely 12 and 12a set part from one another,

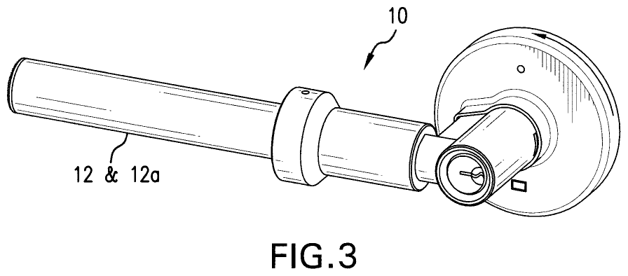

FIG. 3 is a partial perspective view of invention 10 illustrating door tube handles 12 and 12a.

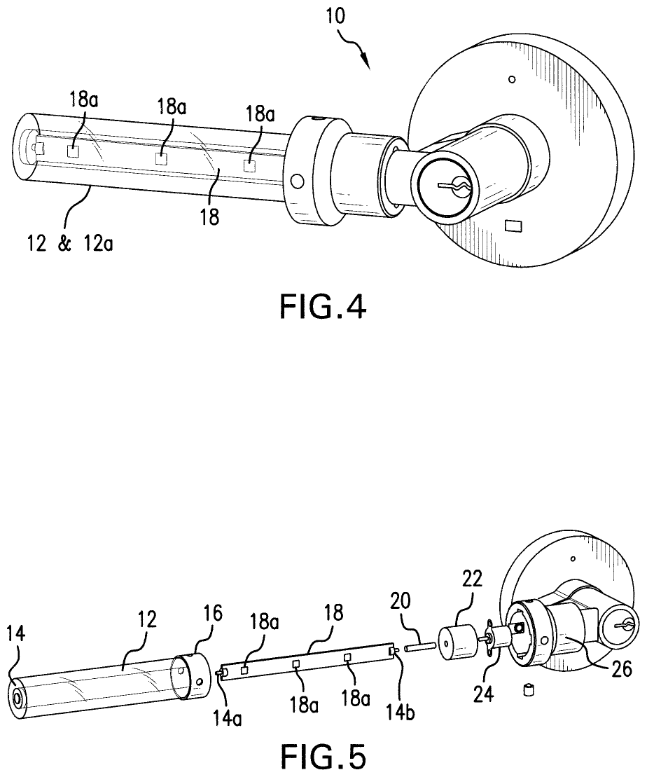

FIG. 4 depicts successive firing UVC LEDs 18a located on rotating circuit board 18 contained in translucent door handle tubes 12 and 12a.

FIG. 5 is a partial exploded view of invention 10 illustrating end plug 14 affixed to one end of translucent door tube handle 12, end plug 14 containing an aperture to accept projection 14a of rotating UVC LED circuit board 18. At the second end of rotating UVC LED circuit board is projection 14b that connects with connector 20, which passes through slip rings 22 which in turn is connected to electric motor 24.

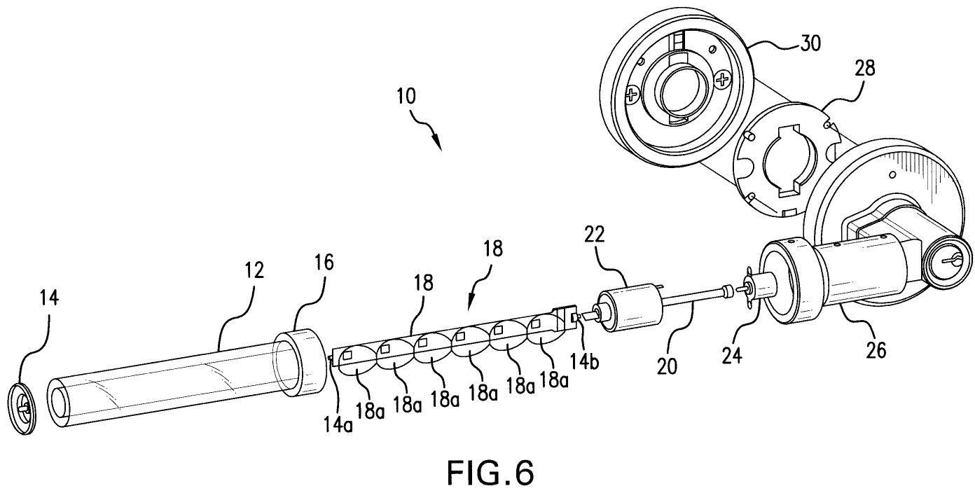

FIG. 6 is a further exploded view of invention 10 illustrating coupler adaptor housing 26 that contain system components. Also shown is control board 28 and power control frame 30.

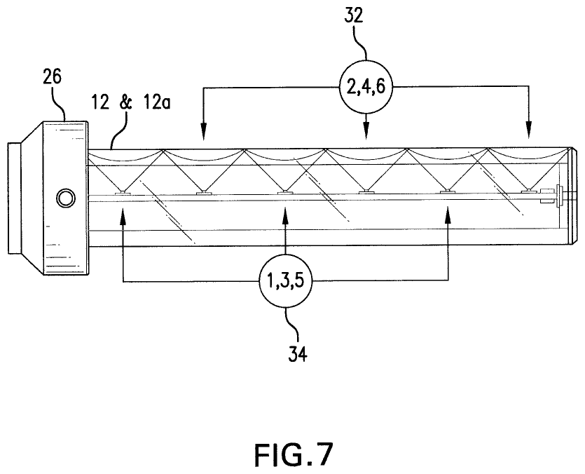

FIG. 7 illustrates rotating successive firing from UVC LED light source 18a within translucent door tube handle 12. As shown 32 indicates firing of UVC LED numbers 2, 4 and 6 firing and then successively at 34 firing of UVC LED numbers 1, 3 and 5. Any number of UVC LED light sources may be utilized on rotating UVC LED circuit board 18. If desired successive firing of the LEDs permits conservative peak amperage demands, that is a door lever with 6 UVC LEDs than requires 4 watts each or a total of 24 watts and by using 2 “strings” of LEDs permits energizing 3 LEDs at a time and subsequently and the other 3 LEDs. Accordingly only 12 watts are needed at any time and is particularly useful for limiting volume of power needed at any time when using batteries as the electrical energy source and the rotation permits an even, economical and uniform dispersion of the UVC LED light.

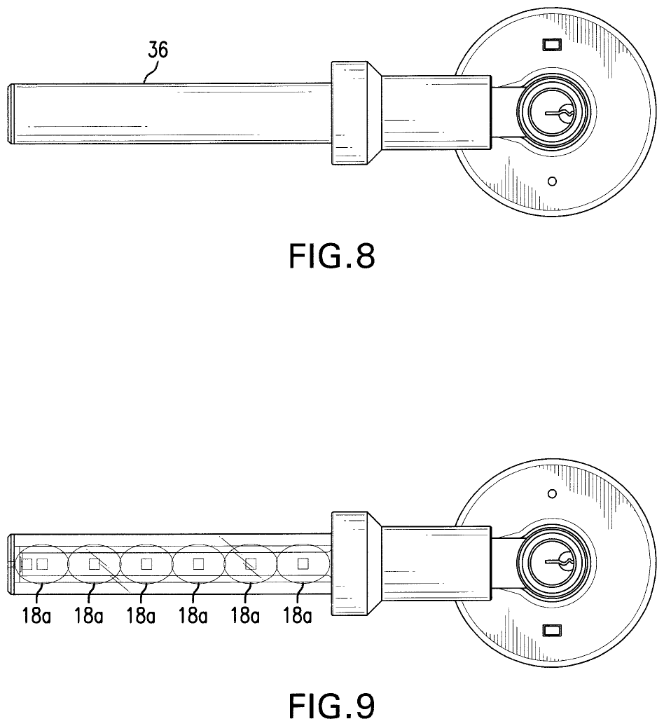

FIG. 8 illustrates a frosted, clear and/or translucent quartz tube 36 in conjunction with invention 10.

FIG. 9 depicts UVC LED light projections 38 contained within door handle tube 12.

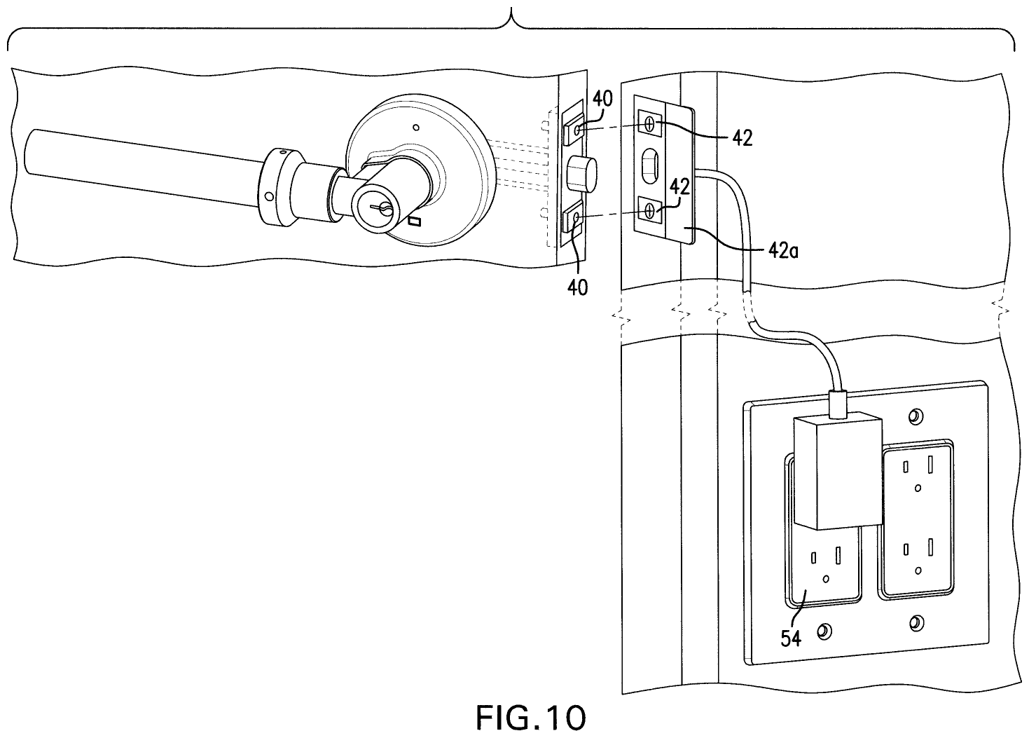

FIG. 10 illustrates the apparatus for supplying electrical power (commonly 120-volt AC) 54 to electrical contacts 40 on door handle latch assembly 10. Electrical conduit 52 on door jamb 10a connects to electrical contacts 42 in door jamb so that electric power is transmitted when contacts 40 and 42 contact each other. Electrical distribution junction 42a is contained in door jamb in wall 10a.

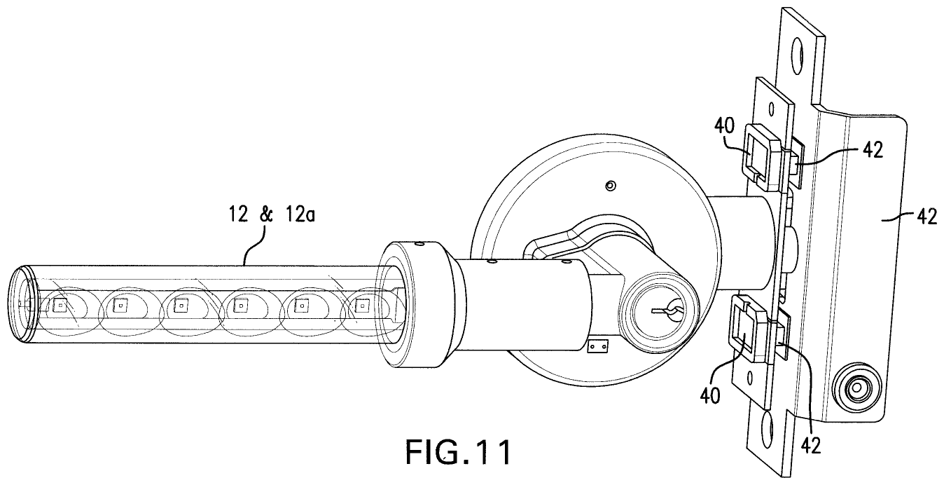

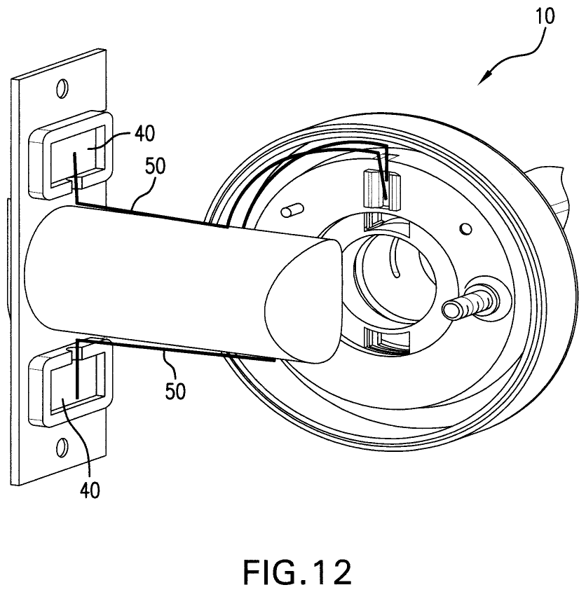

FIG. 11 shows a single translucent door tube handle 12 in contact with electrical distribution junction 44a in door jam in wall 10a and electrical contacts 40 on door handle assembly are in contact with electrical contacts 42 in electrical distribution junction 42a and accordingly the rotating successive firing emitting translucent ultraviolet C self-sterilizing germicidal light tube is energized.

FIG. 12 illustrates electrical contacts 40 connected by electrical conduits 50 to door handle latch assembly 10.

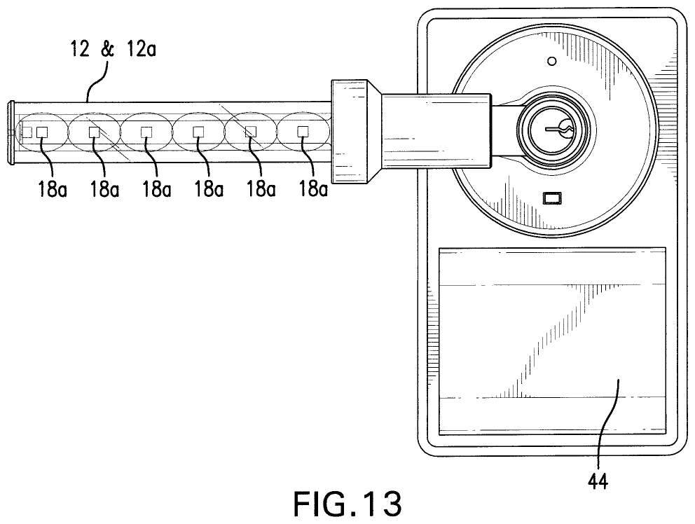

FIG. 13 depicts battery cover 44 as an alternate power source to rotate door tube handle 12 and 12a and also to supply electrical energy to successive firing of UVC LEDs 32 and 32a.

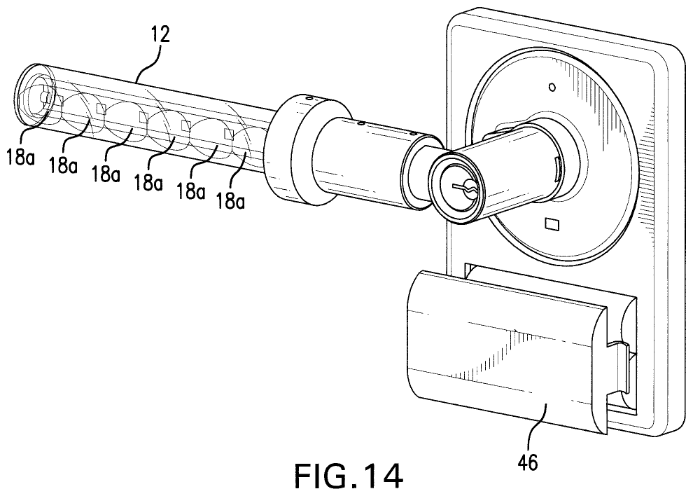

FIG. 14 depicts battery 44 with its cover 46 removed.

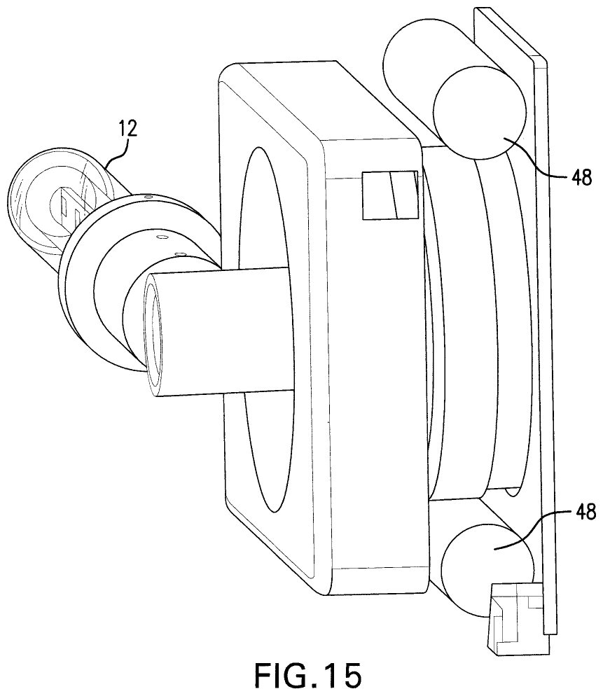

FIG. 15 illustrates batteries 48 affixed to invention 10 and translucent door tube handle with internal rotating UVC LEDs 12.

While only several embodiments of the present invention have been shown and described, it will be obvious to those skilled in the art that many modifications may be made to the present invention without departing from the spirit and scope thereof.

1. A rotating successive firing emitting translucent ultraviolet C self-sterilizing germicidal light tube for a door handle or door lever access device for hand sanitation, comprising:
a tube handle attached to a door latch assembly;
a rotating UVC LED light source contained within said tube handle;
electrical energy for energizing said UVC LED light source;
electrical energy for rotating said UVC LED light source contained within said tube handle; and,
means for rotating said UVC LED light source contained within said tube handle.

2. The rotating successive firing emitting translucent ultraviolet C self-sterilizing germicidal light tube for a door handle or door lever access device for hand sanitation according to claim 1, wherein said means for rotating said UVC LED light source contained within the tube handle is an electric motor.

3. The rotating successive firing emitting translucent ultraviolet C self-sterilizing germicidal light tube for a door handle or door lever access device for hand sanitation according to claim 1, wherein said UVC LED light source contained within said tube handle comprises at least one UVC LED mounted on a circuit board.

4. The rotating successive firing emitting translucent ultraviolet C self-sterilizing germicidal light tube for a door handle or door lever access device for hand sanitation according to claim 1, wherein said rotating UVC LED light source contained within said tube handle is successively firing.

5. The rotating successive firing emitting translucent ultraviolet C self-sterilizing germicidal light tube for a door handle or door lever access device for hand sanitation according to claim 1, wherein said tube handle is fabricated from quartz.

6. The rotating successive firing emitting translucent ultraviolet C self-sterilizing germicidal light tube for a door handle or door lever access device for hand sanitation according to claim 1, wherein said tube handle is translucent.

7. The rotating successive firing emitting translucent ultraviolet C self-sterilizing germicidal light tube for a door handle or door lever access device for hand sanitation according to claim 1, wherein said rotating UVC LED light source contained within the tube handle is attached to a rotating circuit board.

8. The rotating successive firing emitting translucent ultraviolet C self-sterilizing germicidal light tube for a door handle or door lever access device for hand sanitation according to claim 1, wherein said electrical energy for energizing said UVC LED light source is 120-volt AC power.

9. The rotating successive firing emitting translucent ultraviolet C self-sterilizing germicidal light tube for a door handle or door lever access device for hand sanitation according to claim 1, wherein said electrical energy for energizing said UVC LED light source is provided by a battery.

10. The rotating successive firing emitting translucent ultraviolet C self-sterilizing germicidal light tube for a door handle or door lever access device for hand sanitation according to claim 1, wherein said electrical energy for rotating said UVC LED light source contained within said tube handle is 120-volt AC power.

11. The rotating successive firing emitting translucent ultraviolet C self-sterilizing germicidal light tube for a door handle or door lever access device for hand sanitation according to claim 1, wherein said electrical energy for rotating said UVC LED light source contained within said tube handle is provided by a battery.
